(19) United States
(12) Patent Application Publication
(10) Pub. No.: US 2005/0038356 A1
(43) Pub. Date: Feb. 17, 2005

(54) Medical strip electrode

(75) Inventors: Jurgen Fortin, Graz (AT); Winfried Nessler, Munchen (DE); Bernhard Nessler, Innsbruck (AT); Falko Skrabal, Graz (AT)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignees: CNSYSTEMS MEDIZINTECHNIK GMBH; NESSLER MEDIZINTECHNIK GMBH

(21) Appl. No.: 10/660,002

(22) Filed: Sep. 11, 2003

Related U.S. Application Data
(63) Continuation of application No. PCT/AT02/00081, filed on Mar. 21, 2002.

(30) Foreign Application Priority Data
Mar. 13, 2001 (AT) A 392/2001

(51) Int. Cl. A61B 5/0408

(52) U.S. Cl. 600/582

(57) ABSTRACT

A medical electrode is provided for measuring the electrical resistance of the body of a patient, especially an impedance cardiography electrode. The electrode includes a non-conductive, unilaterally adhesive support (1) that is elongated and that forms a connecting strap (1′) at its one end for connecting the electrode with electrical terminals; and two contact strips (2a, 2b) from an electrically conductive aluminum composite film, as the electrode material, that are adhered to the support (1) on the adhesive face thereof. The contact strips (2a, 2b) on their side facing away from the support (1) form a composite structure with a skin-friendly electrically conductive adhesive, leaving free the connection straps or lugs (2a′, 2b′), and the strips bend into connection straps (2a′2b′) at the connecting end of the support (1). The electrode may optionally have a peelable, protective cover for use on the adhesive faces of the support (1) and the contact strips (2a, 2b) that are to come in contact with the body of the patient.

### CROSS-REFERENCE TO RELATED APPLICATIONS

This application is a continuation of International Application No. PCT/AT02/00081, filed Mar. 12, 2002, the disclosure of which is incorporated herein by reference.

### BACKGROUND OF THE INVENTION

The invention relates to medical strip electrodes, particularly impedance cardiography electrodes.

Impedance cardiography is a relatively new field of medical diagnosis and is based on the measurement of the electrical resistance (impedance) of the human body to electrical current when an alternating current is supplied. A range of indicators, such as heartbeat volume, heart output, systemic vessel resistance, speed and acceleration of blood circulation, heart pump output, heart contraction, etc., can be derived from the calculated change in impedance when a defined alternating current is passed through a patient's chest and the voltage changes due to changes in the bloodstream within the field are measured.

Various measurement arrangements are known in the present art for impedance cardiography. One method relied on electrodes that were implanted using pulmonary artery catheters, but was superseded by considerably more practical, non-invasive methods that do not require the implantation of a catheter, one of which consists in applying two annular electrodes, e.g., made from silver or aluminum bands, in each position, completely encircling the body around the neck and below the chest (underneath the ribcage).

However, this is impractical because:

the patient's body must inevitably be moved when the electrodes are applied, which represents a significant danger for the person concerned in the case of bodily injury;

the electrodes are displaced easily when the patient moves and can become detached from the skin;

the patient's breathing is constricted and made difficult by the circular electrodes;

the electrodes must be cut from a continuous roll in the correct length for the corresponding circumference at each body site;

the electrical connections must be stuck or soldered to the metal bands in a separate step as soon as they are the right length;

it is not possible to create a constantly homogeneous field that allows even minor changes in impedance to be measured reliably, since the distance between the two bands in a pair is not defined and not constant; and

the costs of the electrode material and the labor are prohibitive.

A modification of this method provides for discrete electrodes about the neck and below the ribcage instead of the ring electrodes. In this method, for example, conventional, circular EKG electrodes may be used. Then, electrodes are applied in pairs adjacently in the axial direction of the body (i.e., when the patient is standing, they are located one above the other). Here too, one electrode pair at the neck and at least one pair below the thorax are required, though in most cases two electrode pairs are applied opposite one another on either side of the body (that is a total of 8 electrodes), in order to improve the homogeneity of the field. The “outer” electrodes (i.e., the top electrodes at the neck and the bottom electrodes under the ribcage) serve to generate the field, while electrodes lying within the field at each site are used to measure the potential (see FIG. 1 and the explanation in the following).

This method yields better results than the method using the ring electrodes, and is also simpler and less expensive to carry out, since the patient's body does not need to be moved to allow the electrodes to be applied, the electrodes are prefabricated, i.e., they do not have to be adapted (cut) to the size of the body, and they are readily available in the form of commercially available EKG electrodes.

However, the disadvantage of this mode of carrying out impedance cardiography resides precisely in this prefabrication, or more specifically in the electrode geometry. The targeted current in this case is also not capable of creating a field that is sufficiently homogeneous to allow detectable and significant signals to be obtained for minor changes in impedance. A large fraction of diagnostically valuable information cannot be differentiated from the unavoidable background noise. Reproducibility also suffers due to the use of pairs of discrete electrodes, since the distance between the electrodes in a pair is not the same for all measurements, which leads to variations in the measurement results. Even when the two electrodes in a pair are connected (e.g., by prior fabrication of a pre-stamped foil in a “figure 8”), the possibility remains that this eight is not applied parallel to the axis of the body, with unfortunate consequences for the reproducibility of the measurement results.

### BRIEF SUMMARY OF THE INVENTION

An objective of the invention is therefore to provide new electrodes with which the described disadvantages of the prior art may be overcome.

This objective is achieved according to the invention with a medical electrode for measuring the electrical impedance in the bodies of patients, particularly an impedance cardiography electrode, including the following components:

an elongated, electrically non-conductive support surface, adhesive on one side, one end of which has the form of a connecting strap for attaching the electrode to electrical connections;

two contact strips made from electrically conductive aluminum composite film as the electrode material, that are stuck to the support surface on the adhesive side thereof, and that form a bonding structure with an electrically conductive, skin-compatible adhesive, with recessing of the connection straps on the surfaces facing away from the support surface, and which are also shaped at the connection end of the support surface into respective connection straps; and

optionally a removable protective covering for the adhesive surfaces of the support surface and the contact strips that come into contact with the patient's body during use.

When such electrodes according to the invention are used, a homogeneous electrical field is assured since the current is applied not topically, but over a longer section (perpendicular to the axis of the body), while the patient does not have to be moved for the electrodes to be attached. The electrode material itself is a composite of an aluminum foil with a skin-compatible, electrically conductive adhesive, i.e., the electrode material is stuck directly to the skin, so that contact with the patient's body is assured over the entire length of the electrode, thereby ensuring that it will not become detached.

The provision of a two-part electrode of such kind also guarantees a constant separation between the two strip-shaped contacts, which enables the best possible reproducibility. Moreover, only two or three electrodes according to the invention need to be attached to the body in order to create the electrical field, instead of the eight electrodes required in the prior art, since both the stomach and the neck electrodes may extend from one side of the body to the other, which in turn also reduces the number of connections and thus also wires, thereby also rendering the system more manageable.

Except in the section where they assume the shape of connection straps, the two contact strips preferably extend essentially parallel with a separation of about 15 to 50 mm, preferably about 20 to 40 mm, particularly about 25 to 30 mm, and have a width of about 3 to 10 mm, preferably about 4 to 7 mm, particularly about 5 mm. The length of the contact strips is preferably in a range from about 50 to 500 mm, particularly about 100 to 400 mm, more preferably about 150 to 300 mm, and especially about 200 mm.

The minimum separation between the contact strips is necessary in order to prevent mutual interference and degradation of the alternating field. The maximum separation according to the invention is determined on the basis of cost, since a larger separation would increase manufacturing costs unnecessarily. In determining both length and width, a compromise was struck in order to obtain the largest possible contact length or area while keeping manufacturing costs as low as possible.

The support surface is normally made from non-conductive plastic foam, one side of which is adhesive. The electrically non-conductive adhesive provided on one side thereof is preferably also skin-compatible, since it is in direct contact with the patient's skin during the measurement.

In a preferred embodiment, the geometry of the connection straps corresponds with that of standardized, commercially available electrical connections, e.g., for a terminal clip for neutral electrodes used in HF surgery, so that there is no manufacturing expense for special connections for the electrodes according to the invention, which in turn reduces costs and further improves the manageability of the electrodes.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a schematic representation of an impedance cardiography measurement arrangement using eight electrodes according to the prior art;

FIG. 2 is a schematic representation of an impedance cardiography measurement arrangement similar to that in FIG. 1, but using only three of the electrodes according to the invention; and

FIG. 3 is a fabrication drawing of a preferred embodiment of the electrode according to the invention.

### DETAILED DESCRIPTION OF THE INVENTION

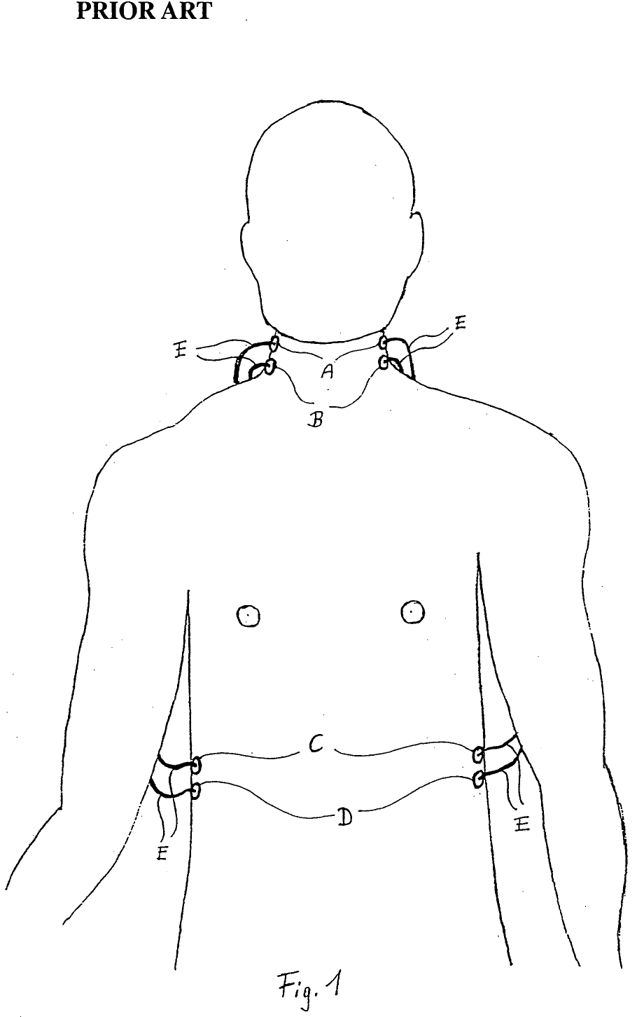

FIG. 1 shows a schematic illustration of the upper body of a patient who is to undergo an impedance cardiography measurement procedure. Four electrodes, A, B, C and D, each arranged in pairs on either side of the patient's body at the neck and below the ribcage in accordance with the prior art, are attached with the associated connecting wires E. The electrodes may be, for example, the conventional round EKG electrodes whose contact areas are usually circular with a diameter of about 10 to 12 mm.

The two highest and lowest electrodes in FIG. 1 (electrodes A and D) are used to generate the electrical field over the entire chest of the patient, whereas the inner electrode pairs in each case (electrodes B and C) are used to measure the impedance.

According to the prior art, the current is thus applied topically, which means that it is not able to create a homogeneous electrical alternating field between the (in this case four) spot electrodes that is strong enough to allow sensitive measurements to be made.

In medical practice (e.g., with the use of BioZ®, and BioZ.como® brand systems produced by CardioDynamics), the field is created by applying an alternating current at a frequency of 70 kHz and a strength of 2.5 mA. ISO or EN (no. 60-601-1) standards permit current strengths of up to 4 mA for “body flow” certification. According to the prior art, the detection limit for potential measurement after amplification, integration and digital enhancing of the signals is in the order of 0.1 to 1 μV.

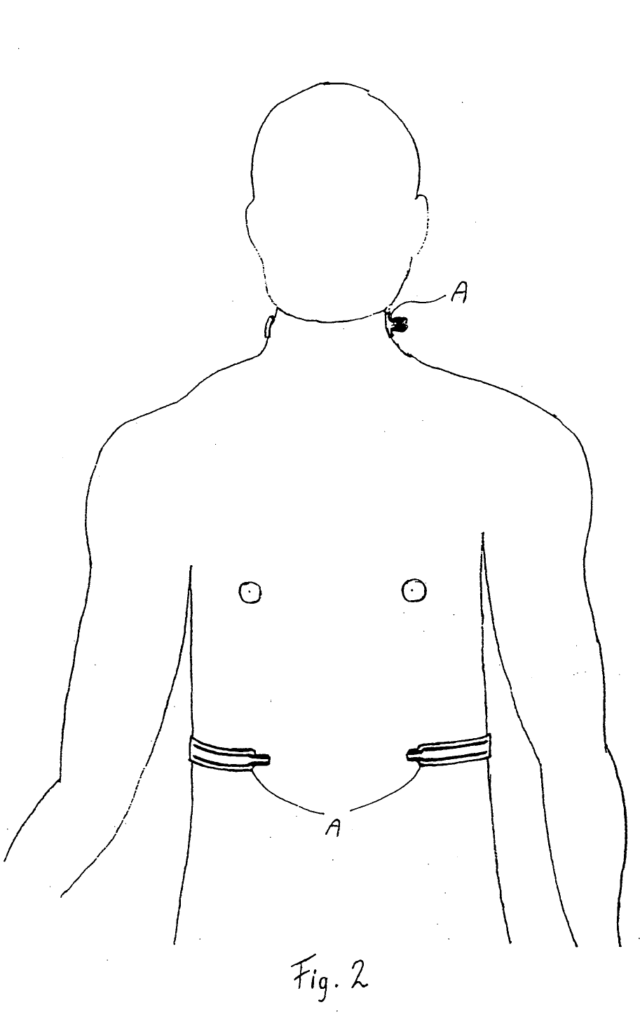

By way of comparison, FIG. 2 is a schematic representation of a measurement arrangement using the electrodes according to the invention. In FIG. 2 an electrode A according to the invention is attached to the back of the neck (therefore only the ends thereof are visible), extending from one side of the neck to the other, this being the reason why a second neck electrode is not required. Two more electrodes A having the same construction are attached, i.e., adhered at the same level, below the chest. In this sketch, the division of the electrode material into two contact strips is indicated schematically, particularly in the two lower electrodes, although in practice of course these would be obscured by the support surface.

The cable connections do not adhere to the body, as may be seen particularly clearly in the case of the neck electrode, and they are connected for measurement purposes to the power source and the measurement device via standard terminal clips (not shown), such as are used, for example, for neutral HF surgery electrodes. This means that a special terminal clip does not need to be developed for the electrodes according to the invention.

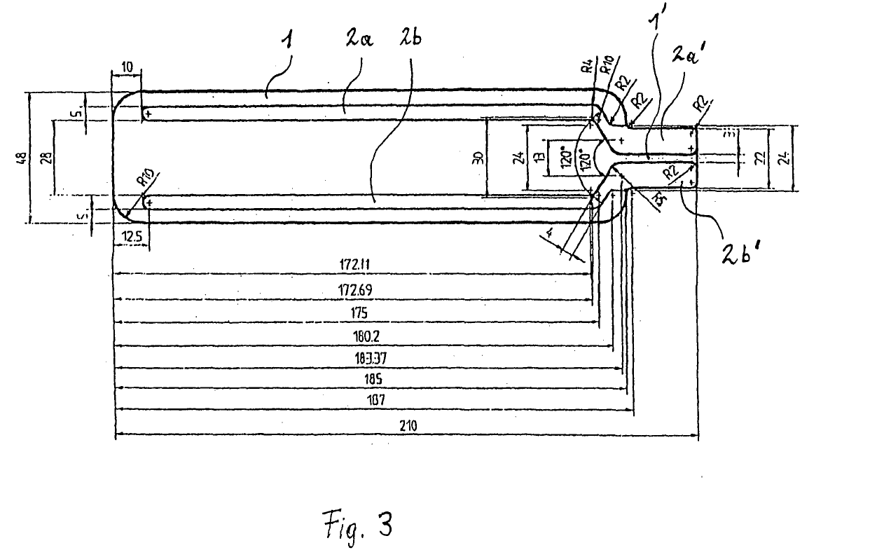

As is shown in FIGS. 1 and 2, the electrode surfaces of the contact strips according to the present invention are considerably larger than those of the prior art and are designed (see FIG. 3) to extend in any case parallel to and at a constant distance from one another. Moreover, medical staff (e.g., nurses) are more easily able to stick elongated electrodes correctly, i.e., at right angles to the axis of the body and at the same height as shown in FIG. 2, than to correctly position the much smaller EKG electrodes shown in FIG. 1 (parallel to the axis of the body and at the same height), even if those shown in FIG. 1 were to be connected in a “figure 8” (for which an extra, separate manufacturing step would be required and the EKG electrodes could no longer be used directly).

According to the present invention, it is also possible to use only two electrodes, i.e., one on the neck and one on the ribcage. The lower electrode may then be the same length as the neck electrode, or also longer, e.g., about 500 mm long, so that it extends across the entire stomach. The former configuration is less favored for purposes of creating the most homogeneous electrical field possible; the latter is less favored for financial reasons, since it is significantly less expensive to produce only one (shorter) construction of the electrode according to the invention. Thus, FIG. 2 represents the preferred compromise according to the invention between field homogeneity and production cost, with the use of three electrodes A having a length of about 200 mm, which corresponds approximately with half the circumference of the neck of an adult.

In FIG. 3, this preferred embodiment of the present invention is shown as a top view fabrication drawing. Two contact strips 2a, 2b are arranged on a support surface 1 having a length of 210 mm. Support surface 1 is preferably made from a foam, such as is normally used for medical electrodes, so that the material may be manufactured inexpensively, is soft and flexible, and adapts well to the contours of the body. The top side (facing the observer) of the support surface is adhesive, i.e., provided with an electrically non-conductive, preferably skin-compatible adhesive, e.g., gel, and contact strips 2a, 2b are bonded with support surface 1 so tightly by the adhesive that they cannot be detached from support surface 1 while the electrode is being manipulated, and particularly when these are removed.

Contact strips 2a, 2b are made in this case from a composite material of an aluminum and a stabilizing plastic foil, together with a skin-compatible, electrically conductive adhesive on the aluminum side, which serves to stick to the skin, while the other, plastic side of the composite foil is stuck firmly to support surface 1.

The total length of support surface 1 is 210 mm; that of contact strips 2a, 2b in this embodiment is 200 mm; i.e., the support surface protrudes beyond contact strips 2a, 2b by 10 mm at one end, which—in addition to the adhesive effect of the contact strips themselves—prevents the contact strip ends from becoming detached from the skin.

The total width of support surface 1 is 48 mm; the width of contact strips 2a, 2b is 5 mm over most of their length. A width of less than 3 mm is not favored for contact strips 2a, 2b, since the homogeneity of the field is degraded unacceptably if the contact strips are too narrow, whereas a width greater than 10 mm raises production costs unnecessarily, since it does not significantly improve field stability. The preferred range is about 4 to 7 mm, and particularly about 5 to 6 mm has been shown to be optimum.

Support surface 1 protrudes beyond contact strips 2a, 2b by 5 mm along the longitudinal edge of the electrode, which also serves to further enhance the attachment of the contact strips.

Contact strips 2a, 2b extend parallel to one another and with a defined separation of (in this embodiment) 28 mm for most of the length of the electrode. The minimum separation for avoiding mutual interference between the contacts is about 15 to 20 mm; the maximum practical separation with consideration for financial constraints is about 50 mm. A separation of 28 to 30 mm was determined to represent the optimum compromise between interference and material cost considerations.

One end of each of the support surface 1 and contact strips 2a and 2b is shaped into a connection strap or lug 1′, 2a′ and 2b′, the width of support surface 1 being reduced (from 48 mm to 22 mm), while contact strips 2a, 2b are widened (from 5 mm to 9.5 mm), and their separation from one another is reduced (from 30 mm to 3 mm). In this way, the strap or lug formed thereby may be attached to a conventional terminal clip, and the design of a special clip becomes unnecessary, thus reducing costs.

In order to prevent adhesion to the terminal clip during measurement, the contact strips are non-adhesive in the area of the connection straps.

The top side of support surface 1 shown in FIG. 3 and contact strips 2a, 2b are protected in storage by a conventional tear-off foil (not shown) to prevent the contacts and adhesive surfaces from dirt and damage. This foil must be removed before use.

With such electrodes according to the invention, it is possible to create an alternating field over the patient's chest that is considerably more homogeneous and stable than with the prior art, with the result that the sensitivity, reproducibility and precision of the measurements are significantly increased.

For example, with the measuring arrangement shown in FIG. 2, it is possible to work with an alternating field frequency of only 40 kHz and a current strength of only 350 μA. This enables the detection threshold of the measured voltage signals to be lowered to the range of 0.01 μ, while the measurement values are available within a few seconds. By contrast, according to the prior art, it was necessary to wait up to a minute before a clear signal was obtained.

Moreover, a measuring arrangement of such kind using the electrodes according to the invention is certifiable for “cardiac flow (CF)” applications, for which a current strength not exceeding 0.4 mA may be applied according to ISO or EN standards (no. 60-601-1), which means that impedance may be measured with electrodes according to the present invention, for example, even during open heart surgery.

The present invention thus provides new medical electrodes, particularly electrodes for impedance cardiography, that offer the following advantages over the prior art:

1) the patient's body does not need to be moved to enable the electrodes to be attached;

2) the electrodes are prefabricated, i.e., the electrodes do not need to be cut to size and the contacts do not need to be soldered;

3) only two or three electrodes according to the invention are needed, which makes handling easier and attachment faster;

4) the adhesion of the electrodes in the correct alignment, i.e., parallel to each other and at the same height and at right angles to the axis of the body, is significantly easier, which facilitates reproducibility;

5) the contact strips of the electrodes according to the invention are stuck directly to the patient's body, so that slipping is prevented;

6) a more stable, homogeneous alternating electrical field may be created, which permits far more precise measurements;

7) the measurement range may be reduced by at least an order of ten, so that not only is precision increased, but also “CF” certification for measuring, even during open heart surgery, may be attained; and

8) costs may be reduced significantly, since inexpensive Al is used as the electrode material instead of Ag, in preferred embodiments of the invention only one electrode shape needs to be produced, the skin does not have to be treated beforehand with conductive gel, and conventional terminal clips may be used.

The reduced manufacturing costs and excellent reproducibility of measurements that result from this simpler handling mean that the commercial applicability of the electrodes according to the invention is not in doubt.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A medical electrode for measuring electrical impedance in a patient body, comprising:
an elongated, electrically non-conductive support surface (1), which is adhesive on one side, and one end of which has a form of a connecting strap (1′) for attaching the electrode to electrical connections;
two contact strips (2a, 2b), electrically insulated from each other, comprising electrically conductive aluminum composite foil as the electrode material, the contact strips being adhered to the support surface (1) on the adhesive side thereof, forming a bonding structure with an electrically conductive, skin-compatible adhesive with recessing of connection straps (2a′, 2b′) on surfaces of the contact strips facing away from the support surface (1), and being shaped at a connection end of the support surface (1) into respective connection straps (2a′, 2b′); and
a removable protective covering for the adhesive surfaces of the support surface (1) and the contact strips (2a, 2b) that come into contact with the patient body during use;
wherein a length of the contact strips (2a, 2b) is in a range of about 50 to 600 mm.

2. The electrode according to claim 1, wherein, except where the contact strips (2a, 2b) are shaped into connection straps (2a′, 2b′), the two contact strips (2a, 2b) extend essentially parallel to one another with a separation of about 15 to 50 mm and have a width of about 3 to 10 mm.

3. The electrode according to claim 1, wherein, except in an area where the contact strips are shaped into connection straps (1′, 2a′, 2b′), a separation distance between outer edges of the contact strips (2a, 2b) and an outer edge of the support surface (1) is about 1 to 20 mm.

4. The electrode according to claim 1, wherein the support surface (1) comprises electrically non-conductive plastic foam that is adhesive on one side.

5. The electrode according to claim 4, wherein the adhesive on the one side of the support surface (1) is skin-compatible.

6. The electrode according to claim 1, wherein a geometry of the connection straps (1′, 2a′, 2b′) matches that of standardized, commercially available electrical connections.

7. The electrode according to claim 1, wherein the electrode is an impedance cardiography electrode.

8. The electrode according to claim 1, wherein the length of the contact strips (2a, 2b) is in a range of about 100 to 400 mm.

9. The electrode according to claim 8, wherein the length of the contact strips (2a, 2b) is in a range of about 150 to 300 mm.

10. The electrode according to claim 8, wherein the length of the contact strips (2a, 2b) is about 200 mm.

11. The electrode according to claim 2, wherein the contact strips (2a, 2b) have a separation of about 20 to 40 mm and have a width of about 4 to 7 mm.

12. The electrode according to claim 11, wherein the contact strips (2a, 2b) have a separation of about 25 to 30 mm and have a width of about 5 mm.

13. The electrode according to claim 3, wherein the separation distance is about 3 to 15 mm

14. The electrode according to claim 13, wherein the separation distance is about 4 to 12 mm.

15. The electrode according to claim 3, wherein the separation distance along longitudinal edges of the electrode is about 5 mm and the separation distance at the end of the electrode opposite the contact strips (1′, 2a, 2b) is about 10 mm.

16. The electrode according to claim 6, wherein the geometry matches that for a terminal clip of neutral electrodes for HF surgery.
